(19) United States
(12) Patent Application Publication
(10) Pub. No.: US 2015/0119297 A1
(43) Pub. Date: Apr. 30, 2015

(54) SYSTEM AND METHOD FOR PROCESSING GENOTYPE INFORMATION RELATING TO DRUG METABOLISM

(71) Applicant: Brian Meshkin, (US)

(72) Inventor: Brian Meshkin, Fulton, MD (US)

(21) Appl. No.: 14/065,072

(22) Filed: Oct. 28, 2013

(51) Int. Cl. G06F 19/00; C12Q 1/68

(52) U.S. Cl. CPC G06F 19/3487; C12Q 1/6883; C12Q 2600/118; C12Q 2600/106
USPC 506/38; 506/39

(57) ABSTRACT

There are systems and methods for performing an assay to generate genotype information about a subject associated with a medical condition. There are also systems and method for generating and utilizing prognostic information associated with treating the patient with a medication based on the genotype information and an association of the genotype and metabolizing the medication. The genotype information includes data relating to SNP alleles in the patient's genotype and the association of the alleles and metabolism of a medication by the patient.

### BACKGROUND

In nature, organisms of the same species usually differ from each other in various aspects, e.g., their appearance. The differences are often based on genetic distinctions, some of which are referred to as polymorphisms. A genetic polymorphism is an occurrence in a population of two or more genetically alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism can also play a role in determining differences in an individual's response to drugs. Pharmacogenetics and pharmacogenomics are multidisciplinary research efforts to study the relationships among genotype, gene expression profiles, and phenotype, as often expressed in variability between individuals in response to drugs taken. Since the initial sequencing of the human genome, more than a million single nucleotide polymorphisms (SNPs) have been identified. Through pharmacogenomic studies some of these SNPs have been associated with substantial changes in the metabolism or effects of medications and some have been used to predict clinical response.

Xenobiotics are often pharmacologically, endocrinologically or toxicologically active substances foreign to a biological system. Many xenobiotics, including pharmacologically active molecules, are commonly lipophilic and often remain non-ionized or partly ionized at physiological pH. The primary purpose of xenobiotic metabolism is to enzymatically convert a lipid-soluble xenobiotic into polar, water soluble and excretable metabolites that can be eliminated. It can also convert prodrugs into therapeutically active compounds and other metabolites. Pharmacological drug (xenobiotic) metabolism pathways are commonly classified as either Phase I or Phase II reactions. Phase I reactions are functionalization reactions (i.e., oxidation, reduction and hydrolysis) in which a derivatizable group is added to the original molecule. Functionalization commonly prepares the drug product molecule for further metabolism in Phase II reactions. Phase II metabolism commonly involves enzyme-catalyzed conjugation of xenobiotics with groups such as glucoronic acid, sulphate and glutathione. The effect of Phase II reactions is often to increase water solubility, thus aiding excretion of the drug metabolites in urine or bile.

In humans, the cytochrome P-450 (CYP) enzymes, a superfamily of microsomal drug-metabolizing enzymes, commonly play a central role in Phase I drug metabolism and are often involved in drug interactions and inter-individual variability in drug metabolism. Members of three CYP gene families, CYP1, CYP2 and CYP3 are known to be associated with drug metabolism. The liver is the major site of activity for these enzymes; however CYP enzymes are also expressed in other tissues.

It is well recognized that different patients respond in different ways to the same medication. The existence of large population differences with small intra-patient variability suggests the role of genetics in determining drug response. Although many non-genetic factors, such as age, nutritional status, renal and liver function and concomitant therapy; often influence the effects of medications it is estimated that genetic differences commonly account for a substantial amount of variability in drug disposition and effects.

In pharmacogenomics, there is a desire to identify new polymorphisms and/or haplotypes to increase the sensitivity of genetic testing. Of special interest is testing for determining genotype-based dose adjustments. The genotype-based information may help a prescriber understand why a patient is or is not responding to an average dose. The may also help a prescriber decide between medications based on a genetic predisposition to drug-metabolizing enzyme activity, such as enzymes that are associated with the CYP450 gene. There is also a desire for methods for predicting and/or diagnosing individuals exhibiting irregular metabolism activity. Furthermore, there is also a desire to detect novel alleles and/or haplotypes for predicting variations in drug metabolism among individuals and to implement this genetic information in a systematic way. Such genetic information can be very useful in providing prognostic information about treatment options for a patient.

Given the foregoing, and to address the above-described limitations, systems and methods are desired for identifying, estimating and/or determining a potential for success of an individual's clinical outcome based on their genetic variations associated with drug metabolism.

### SUMMARY

This summary is provided to introduce a selection of concepts that are further described in the Detailed Description below. The genes, polymorphisms, sequences and sequence identifiers (i.e., SEQ IDs and/or SEQ ID NOs) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with drug metabolism in a patient. The prognostic information is derived from genotype information about a patient's genotype. The genotype information may be obtained by, inter alia, assaying a sample of genetic material associated with a patient.

The systems, methods and CRMs, according to the principles of the invention, can be utilized to determine prognostic information associated with a risk to a patient if they are administered a drug and their ability to properly metabolize it. The prognostic information may be used for addressing prescription needs directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with drug metabolism in a patient, in accordance with the principles of the invention.

According to a first principle of the invention, there is a system for performing an assay. The system may include a sample interface configured to present a sample of genetic material, for example, human material. The patient may be a patient having and/or associated with having a medical condition, the patient having a genotype. The system may also include a detector. The detector may be configured for detecting in the sample a presence of one or more (e.g., any number of 1 to 40) polymorphisms in the genotype to determine an assay result. The assay result may include data describing a presence or an absence of the polymorphisms. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The detector may be configured for detecting a presence of any number (e.g., any number of 1 to 40) and/or combination of the polymorphisms in the group. The system may also include a data management module configured to generate, utilizing a processor, genotype information associated with the tested polymorphisms. The system may also include a storage configured to store the generated genotype information, and/or a system interface configured to transmit the generated genotype information. The detector may be configured to utilize a processor. The detector may also be configured to utilize one or more assay methodologies including: allele specific hybridization, allele specific oligonucleotide ligation, primer extension, mini-sequencing, mass spectroscopy, hetero-duplex analysis, single strand conformational polymorphism, denaturing gradient gel electrophoresis, oligonucleotide microarray analysis, temperature gradient gel electrophoresis and combinations thereof. The detector may also be configured to detect for the presence of one or more sequences including: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a DNA fragment thereof, a homologous DNA sequence thereof having at least 50% homology, a DNA sequence comprising a sequence thereof and combinations thereof.

According to a second principle of the invention, there is a method for performing an assay. The method includes obtaining a sample of genetic material, for example, human material. The patient may be a patient having and/or associated with having a medical condition, the patient having a genotype. The method also includes testing the sample for detecting a presence in the sample of one or more (e.g., any number of 1 to 40) polymorphisms in the genotype to determine an assay result. The assay result may include data describing a presence or an absence of the polymorphisms. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may include generating, utilizing a processor, genotype information associated with the determined assay result. The method may also include storing the generated genotype information; or transmitting the generated genotype information. The method may include testing for detecting for the presence of any number (e.g., 1 to 40) and/or combination of the polymorphisms in the group. The testing may be performed utilizing a processor. The testing may include utilizing one or more methodologies including: allele specific hybridization, allele specific oligonucleotide ligation, primer extension, mini-sequencing, mass spectroscopy, hetero-duplex analysis, single strand conformational polymorphism, denaturing gradient gel electrophoresis, oligonucleotide microarray analysis, temperature gradient gel electrophoresis and combinations thereof. The testing may include detecting for the presence of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a DNA fragment thereof, a homologous DNA sequence thereof having at least 50% homology, and combinations thereof.

According to a third principle of the invention, there is a non-transitory computer readable medium storing computer readable instructions that when executed by a computer system perform a method for generating genotype information. The method includes obtaining a sample of genetic material, for example, human material. The patient may be a patient having and/or associated with having a medical condition, the patient having a genotype. The method also includes testing the sample for detecting a presence in the sample of one or more (e.g., any number of 1 to 40) polymorphisms in the genotype to determine an assay result. The assay result may include data describing a presence or an absence of the polymorphisms. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may include generating, utilizing a processor, genotype information associated with the determined assay result. The method may also include storing the generated genotype information; or transmitting the generated genotype information. The method may include testing for detecting for the presence of any number (e.g., 1 to 40) and/or combination of the polymorphisms in the group. The testing may be performed utilizing a processor. The testing may include utilizing one or more methodologies including: allele specific hybridization, allele specific oligonucleotide ligation, primer extension, mini-sequencing, mass spectroscopy, hetero-duplex analysis, single strand conformational polymorphism, denaturing gradient gel electrophoresis, oligonucleotide microarray analysis, temperature gradient gel electrophoresis and combinations thereof. The testing may include detecting for the presence of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a DNA fragment thereof, a homologous DNA sequence thereof having at least 50% homology, and combinations thereof.

According to a fourth principle of the invention, there is a system for preparing prognostic information. The system includes a receiving interface configured to receive genotype information including data indicating a presence or an absence of one or more (e.g., 1 to 40) polymorphism(s) in a genotype of a patient associated with having a medical condition. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The system may include a data management module configured to generate, utilizing a processor, prognostic information including one or more predictive value(s) associated with the patient and their treatment with a medication associated with addressing their medical condition. The predictive value(s) may correspond with respective polymorphism(s) selected from the group. The receiving interface may be configured to receive genotype information including data indicating the presence or absence of any number (e.g., 1 through 40) and/or combination of the polymorphisms in the group. The data management module may be configured to generate the prognostic information utilizing a scoring function to determine the predictive value(s) based on the indicated presence or absence of the polymorphism(s). The data management module may also be configured to determine the predictive value(s) based on the indicated presence or absence of the polymorphism(s) being homozygous or heterozygous. The data management module may also be configured to generate the prognostic information by adding the predictive value(s) to determine at least one aggregate value(s). The data management module may also be configured to generate the prognostic information by comparing the determined aggregate value(s) with at least one threshold value(s) to determine at least one risk value(s) associated with the patient being treated with the pain medication. The system may also include a storage configured to store the generated prognostic information and/or a transmitting interface configured to transmit the generated prognostic information.

According to a fifth principle of the invention, there is a method for preparing prognostic information. The method includes receiving genotype information including data indicating a presence or an absence of one or more (e.g., 1 to 40) polymorphisms in a genotype of a patient associated with having a medical condition. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may also include generating, utilizing a processor, the prognostic information including one or more predictive value(s) associated with the patient and their treatment with a medication. The predictive value(s) can correspond with respective polymorphism(s) selected from the group. The received genotype information includes data indicating the presence or absence of any number (e.g., 1 through 40) and/or combination of the polymorphisms in the group. The method may utilize a scoring function to determine the predictive values based on the indicated presence or absence of the polymorphisms. The scoring function may determine the predictive values based on the indicated present or absent polymorphisms being homozygous or heterozygous. In the method, generating the prognostic information may include adding the predictive values to determine aggregate values. In the method, generating the prognostic information may include comparing the determined aggregate values with one or more threshold values to determine one or more risk values associated with the patient being treated with the medication.

According to a sixth principle of the invention, there is a non-transitory computer readable medium storing computer readable instructions that when executed by a computer system perform a method for preparing prognostic information. The method includes receiving genotype information including data indicating a presence or an absence of one or more (e.g., 1 to 40) polymorphisms in a genotype of a patient associated with having a medical condition. The polymorphisms may be selected from a group including one or more of: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may also include generating, utilizing a processor, the prognostic information including one or more predictive value(s) associated with the patient and their treatment with a medication. The predictive value(s) can correspond with respective polymorphism(s) selected from the group. The received genotype information includes data indicating the presence or absence of any number (e.g., 1 through 40) and/or combination of the polymorphisms in the group. The method may utilize a scoring function to determine the predictive values based on the indicated presence or absence of the polymorphisms. The scoring function may determine the predictive values based on the indicated present or absent polymorphisms being homozygous or heterozygous. In the method, generating the prognostic information may include adding the predictive values to determine aggregate values. In the method, generating the prognostic information may include comparing the determined aggregate values with one or more threshold values to determine one or more risk values associated with the patient being treated with the medication.

According to a seventh principle of the invention, there is a system for utilizing prognostic information. The system includes a receiving interface configured to receive prognostic information associated with genotype information. The genotype information includes data indicating a presence or an absence of one or more (e.g., 1 to 40) polymorphism(s) in a genotype of a patient associated with having a medical condition. The polymorphisms may be selected from a group including: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The system includes a data management module configured to generate, utilizing a processor, the prognostic information comprising at least one predictive value(s) associated with the patient and their treatment with a medication. The medication may be associated with addressing the patient's medical condition. The predictive value(s) may correspond with one or more of the respective polymorphisms selected from the group. The data management module may be configured to compare the received prognostic information with a medical record associated with the patient. The data management module may be configured to identify a risk value associated with an efficacy and/or a side effect of the medication associated with treating the patient.

According to an eighth principle of the invention, there is a method for utilizing prognostic information. The method may include receiving prognostic information associated with genotype information including data indicating a presence or an absence of one or more polymorphisms in a genotype of a patient associated with having a medical condition. The polymorphisms may be selected from a group including: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may include utilizing the received prognostic information to identify one or more risk value(s) associated with the patient and their treatment with a medication. The medication may be associated addressing the patient's medical condition. The method may include comparing the received prognostic information with a medical record associated with the patient. The method may include utilizing a processor. The method may include identifying risk values associated with an efficacy and/or side effect of the medication associated with treating the patient.

According to a ninth principle of the invention, there is a non-transitory computer readable medium storing computer readable instructions that when executed by a computer system perform a method for utilizing prognostic information. The method may include receiving prognostic information associated with genotype information including data indicating a presence or an absence of one or more polymorphisms in a genotype of a patient associated with having a medical condition. The polymorphisms may be selected from a group including: a T allele at the marker of SEQ ID No: 1 in the CYP2C9 gene, a C allele at the marker of SEQ ID No: 2 in the CYP2C9 gene, a C allele at the marker of SEQ ID No: 3 in the CYP2C9 gene, a G allele at the marker of SEQ ID No: 4 in the CYP2C9 gene, a deletion allele at the marker of SEQ ID No: 5 in the CYP2C9 gene, an A allele at the marker of SEQ ID No: 6 in the CYP2C19 gene, an A allele at the marker of SEQ ID No: 7 in the CYP2C19 gene, a G allele at the marker of SEQ ID No: 8 in the CYP2C19 gene, a T allele at the marker of SEQ ID No: 9 in the CYP2C19 gene, an A allele at the marker of SEQ ID No: 10 in the CYP2C19 gene, an A allele at the marker of SEQ ID No: 11 in the CYP2C19 gene, a C allele at the marker of SEQ ID No: 12 in the CYP2C19 gene, a G allele at the marker of SEQ ID No: 13 in the CYP2D6 gene, a deletion allele at the marker of SEQ ID No: 14 in the CYP2D6 gene, an A allele at the marker of SEQ ID No: 15 in the CYP2D6 gene, a deletion allele at the marker of SEQ ID No: 16 in the CYP2D6 gene, a C allele at the marker of SEQ ID No: 17 in the CYP2D6 gene, a T allele at the marker of SEQ ID No: 18 in the CYP2D6 gene, a deletion allele at the marker of SEQ ID No: 19 in the CYP2D6 gene, a T allele at the marker of SEQ ID No: 20 in the CYP2D6 gene, a C allele at the marker of SEQ ID No: 21 in the CYP2D6 gene, an A allele at the marker of SEQ ID No: 22 in the CYP2D6 gene, an AA allele at the marker of SEQ ID No: 23 in the CYP2D6 gene, a T allele at the marker of SEQ ID No: 24 in the CYP2D6 gene, a AGTGGGCAC allele at the marker of SEQ ID No: 25 in the CYP2D6 gene, a T allele at the marker of SEQ ID No: 26 in the CYP2D6 gene, an A allele at the marker of SEQ ID No: 27 in the CYP2D6 gene, an A allele at the marker of SEQ ID No: 28 in the CYP2D6 gene, a C allele at the marker of SEQ ID No: 29 in the CYP2D6 gene, an A allele at the marker of SEQ ID No: 30 in the CYP2D6 gene, a G allele at the marker of SEQ ID No: 31 in the CYP3A4 gene, an A allele at the marker of SEQ ID No: 32 in the CYP3A5 gene, an A allele at the marker of SEQ ID No: 33 in the VKORC1 gene, a T allele at the marker of SEQ ID No: 34 in the VKORC1 gene, a G allele at the marker of SEQ ID No: 35 in the VKORC1 gene, a C allele at the marker of SEQ ID No: 36 in the VKORC1 gene, a T allele at the marker of SEQ ID No: 37 in the VKORC1 gene, an A allele at the marker of SEQ ID No: 38 in the VKORC1 gene, an A allele at the marker of SEQ ID No: 39 in the CYP1A2 gene, and a C allele at the marker of SEQ ID No: 40 in the CYP3A7 gene. The method may include utilizing the received prognostic information to identify one or more risk value(s) associated with the patient and their treatment with a medication. The medication may be associated addressing the patient's medical condition. The method may include comparing the received prognostic information with a medical record associated with the patient. The method may include utilizing a processor. The method may include identifying risk values associated with an efficacy and/or side effect of the medication associated with treating the patient.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible, such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genetic profile and their metabolism rate for a drug that is associated with treating the patient's medical condition. The prognostic information may also be utilized for determining genotype-based dose adjustments that can help a prescriber understand why a patient is or is not responding to a medication dosage, such as an “average” dose, or that can help a prescriber decide between medications based on a genetic predisposition to drug-metabolizing enzyme activity, such as enzymes associated with the CYP450 gene. The prognostic information may also be utilized for predicting and/or diagnosing individuals exhibiting irregular metabolism activity. Such genetic information can be very useful in providing prognostic information about treatment options for a patient.

The patient may be associated with the medical condition. The patient may also have already been prescribed a medication for treating the medical condition. The present invention has been found to be particularly advantageous for determining a treatment for a patient who may have a regular or irregular metabolism rate for metabolizing a drug. While the present invention is not necessarily limited to such applications, as illustrated through the examples below, various aspects of the invention may be appreciated through a discussion of the various examples using this context.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims.

Throughout this disclosure, various publications, patents and published patent specifications are referenced by an identifying citation. The disclosures of these publications, patents and published patent specifications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S.C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)).

### DEFINITIONS

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on”, “comprises”, “comprising”, “includes”, “including”, “has”, “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known to those of ordinary skill in the art.

The terms “protein”, “polypeptide” and “peptide” are used interchangeably herein when referring to a gene product.

The term “allele”, which is used interchangeably herein with “allelic variant”, refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode”, as it is applied to polynucleotides, refers to a polynucleotide which is said to “encode” a polypeptide. The polynucleotide can be transcribed and/or translated to produce mRNA for the polypeptide and/or a fragment thereof. An antisense strand is the complement of such a polynucleotide, and the encoding sequence can be deduced therefrom.

The term “genotype” refers to the allelic composition of a cell or a gene, whereas the term “phenotype” refers to the detectable outward manifestations of a genotype.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, it includes nucleotide(s) having a uracil base that is uridine.

The terms “oligonucleotide” or “polynucleotide”, or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, U for Uracil, R for A or G, Y for C, T or U, K for G, T or U, and M for A or C.

As used herein, the term “average patient metabolism” is used interchangeably with “average metabolism” and “normal metabolism” and refers to a metabolic rate an average person metabolizes substance(s) under standardized condition(s). The terms are a basis for a comparison with the terms “metabolically upregulating” or “upregulating” which refer to a condition, such as a genetic condition, associated with a metabolic rate that is higher than an average metabolic rate. Similarly, the terms are also a basis for the terms “metabolically downregulating” or “downregulating” which refers to a condition, such as a genetic condition, associated with a metabolic rate that is lower than an average metabolic rate.

As used herein, the terms “drug”, “medicament”, and “therapeutic compound” or “compound” are used interchangeably and refer to any chemical entity, pharmaceutical, drug, and the like that can be used to treat or prevent a disease, illness, conditions, or disorder of bodily function. Compounds comprise both known and potential therapeutic compounds. A compound can be determined to be therapeutic by screening using the screening methods of the present invention. A “known therapeutic compound” or “medicament” refers to a therapeutic compound that has been shown (e.g., through animal trials or prior experience with administration to humans) to be effective in such treatment. Examples of test compounds include, but are not limited to peptides, polypeptides, synthetic organic molecules, naturally occurring organic molecules, nucleic acid molecules, and combinations thereof.

For example, therapeutic compounds or medicaments may include, but are not limited to, amiodarone, diisopyramide, verapamil, propranolol, amlodipine, clonidine, diltiazem, felodipine, guanabenz acetate, isradipine, minoxidil, chloride nicardipine, nifedipine, chloride prazosin, papaverine, carbamazepine, decarbazine, etoposide, lomustine, melphalan, mitomicin, mitoanthrone, procarbazine, taxol and derivatives thereof, alprazolam, bromazepam, diazepam, lorazepam, oxazepam, temazepam, sulpiride, triazolam, alprenolol, atenolol, oxprenolol, pindolol, propranolol, salbutamol, salmeterol, aminone, digitoxinn, digoxin, lanatoside C, medigoxine, aclacinomycins, actinomycins, adriamycins, ancitabines, anthramycins, azacitidines, azaserines, 6-azauridines, bisantrenes, bleomycins, cactinomycins, carmofurs, carmustines, carubicins, carzinophilins, chromomycins, cisplatins, cladribines, cytarabines, dactinomycins, daunorubicins, denopterins, 6-diazo-5-oxo-L-norleucines, doxifluridines, doxorubicins, edatrexates, emitefurs, enocitabines, fepirubicins, fludarabines, fluorouracils, gemcitabines, idarubicins, loxuridines, menogarils, 6-mercaptopurines, methotrexates, mithramycins, mycophenolic acids, nogalamycins, olivomycines, peplomycins, pirarubicins, piritrexims, plicamycins, porfiromycins, pteropterins, puromycins, retinoic acids, streptonigrins, streptozocins, tagafurs, tamoxifens, thiamiprines, thioguanines, triamcinolones, trimetrexates, tubercidins, vinblastines, vincristines, zinostatins, zorubicins, fluoxetine, aripiprazole, clozapine, iloperidone, fluphenazine, ziprasidone, haloperidol, paliperidone, loxapine, molindone, thiothixene, pimozide, perphenazine, risperidone, quetiapine, trifluoperazine, thioridazine, chlorpromazine, olanzapine, clomipramine, amoxapine, nortriptyline, citalopram, duloxetine, trazodone, venlafaxine, amitriptyline, selegiline, escitalopram, maprotiline, fluvoxamine, isocarboxazid, phenelzine, desipramine, tranylcypromine, paroxetine, paroxetine-mesylate, desvenlafaxine, mirtazapine, doxepin, trimipramine, imipramine, imipramine pamoate, protriptyline, bupropion, sertraline, divalproex sodium, lithium carbonate, lamotrigine, lithium citrate, lithium carbonate, gabapentin, carbamazepine, topiramate, oxcarbazepine, lorazepam, buspirone, clonazepam, chlordiazepoxide, oxazepam, clorazepate, diazepam, alprazolam, amphetamine, methylphenidate, methamphetamine, dextroamphetamine, dextroamphetamine, dexmethylphenidate, guanfacine, atomoxetine, lisdexamfetamine and dimesylate.

### Exemplary Embodiments

The biological basis for an outcome in a specific patient following a treatment with medication has not been fully understood and is subject, inter alia, to how the medication is metabolized by the patient. Applicant(s) have determined that select allelic variants associated with select genetic markers can shed light on an expected outcome of a treatment including one or more medication(s) and help to identify the metabolic response of an individual subject to treatment with the medication(s). For example, specific polymorphisms found in particular genes can be used to help identify individuals that are likely to experience a positive response or a non-response to treatment with a medication based on their expected rate of metabolizing the drug.

When a genetic marker or polymorphism is used as a basis for selecting a patient for a treatment described herein, the genetic marker or polymorphism may be measured before and/or during treatment, and the values obtained may be used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one of skill in the art, measurement of a genetic marker or polymorphism in a clinical setting can be an indication that this parameter may be used as a basis for initiating, continuing, adjusting and/or ceasing administration of treatment, such as described herein.

To identify the select alleles which may be utilized, according to the principles of the invention, Applicant(s) identified allelic variants associated with genetic markers in several genes and correlated these findings with genetic predisposition(s) to drug-metabolizing enzyme activity. Accordingly, assaying the genotype at these markers can be used to predict the outcome of treatment with a medication based on the expected metabolism of the drug in the patient. Clinicians prescribing the medication may utilize this prognostic information to improve therapeutic decisions, and to avoid treatment failures.

The allelic variants that Applicant(s) have identified as being active (i.e., the “active” allele of the known allelic variants of a DNA polymorphism) for providing prognostic information, according to the principles of the invention, are catalogued by the National Center for Biotechnology Information (NCBI) in the Reference SNP (i.e., “refSNP”) database maintained by NCBI. The Reference SNP database is a polymorphism database (dbSNP) which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

The allelic variants identified by Applicant(s) are polymorphisms associated with genetic markers in several genes which have been associated with the cytochrome P450 and other enzymatic pathways. These genes include the respective genes encoding the Cytochrome P450 2C9 enzyme (abbreviated CYP2C9), the Cytochrome P450 2C19 protein (abbreviated CYP2C19), the Cytochrome P450 2D6 enzyme (abbreviated CYP2D6), the Cytochrome P450 3A4 enzyme (abbreviated CYP3A4), the Cytochrome P450 3A5 enzyme (abbreviated CYP3A5), the Vitamin K epoxide reductase complex subunit 1 enzyme (abbreviated VKORC1), the Cytochrome P450 1A2 enzyme (abbreviated CYP1A2), and the Cytochrome P450 3A7 enzyme (abbreviated CYP3A7). Alleles which Applicant(s) have identified as “active” for predicting of drug metabolizing activity, according to the principles of the invention, are listed in Table 1 below.

TABLE 1*

| | | | | | Inactive |
|---|---|---|---|---|---|
| No. | NCBI rs# | Gene | DNA Context Sequence for Active Allele** | SEQ ID | Allele(s) |
| 1 | rs1799853 | CYP2C9 | GATGGGGAAGAGGAGCATTGAGGAC[T]GTGTTCAAGAGG | SEQ ID No: 1 | ...[C]... |
| | | | AAGCCCGCTGCCT | | |
| 2 | rs1057910 | CYP2C9 | TGTGGTGCACGAGGTCCAGAGATAC[C]TTGACCTTCTCC | SEQ ID No: 2 | ...[A]... |
| | | | CCACCAGCCTGCC | | |
| 3 | rs56165452 | CYP2C9 | GTGGTGCACGAGGTCCAGAGATACA[C]TGACCTTCTCCC | SEQ ID No: 3 | ...[A/T]... |
| | | | CACCAGCCTGCCC | | |
| 4 | rs28371686 | CYP2C9 | TGCACGAGGTCCAGAGATACATTGA[G]CTTCTCCCCACC | SEQ ID No: 4 | ...[C]... |
| | | | AGCCTGCCCCATG | | |
| 5 | rs9332131 | CYP2C9 | GATTGCTTCCTGATGAAAATGGAG[-]AGGTAAAATGTAA | SEQ ID No: 5 | ...[A]... |
| | | | ACAAAAGCTTAGT | | |
| 6 | rs4244285 | CYP2C19 | TTCCCACTATCATTGATTATTTCCC[A]GGAACCCATAAC | SEQ ID No: 6 | ...[C/G]... |
| | | | AAATTACTTAAAA | | |
| 7 | rs4986893 | CYP2C19 | ACATCAGGATTGTAAGCACCCCCTG[A]ATCCAGGTAAGG | SEQ ID No: 7 | ...[G]... |
| | | | CCAAGTTTTTTGC | | |
| 8 | rs28399504 | CYP2C19 | GTCTTAACAAGAGGAGAAGGCTTCA[G]TGGATCCTTTTG | SEQ ID No: 8 | ...[A]... |
| | | | TGGTCCTTGTGCT | | |
| 9 | rs56337013 | CYP2C19 | CCTATGTTTGTTATTTTCAGGAAAA[T]GGATTTGTGTGG | SEQ ID No: 9 | ...[C]... |
| | | | GAGAGGGCCTGGC | | |
| 10 | rs72552267 | CYP2C19 | CGGCGTTTCTCCCTCATGACKCTGC[A]GAATTTTGGGAT | SEQ ID No: 10 | ...[G]... |
| | | | GGGGAAGAGGAGC | | |
| 11 | rs72558186 | CYP2C19 | TGCTTCCTGATCAAAATGGAGAAGG[A]AAAATGTTAACA | SEQ ID No: 11 | ...[T]... |
| | | | AAAGCTTAGTTAT | | |
| 12 | rs41291556 | CYP2C19 | AATCGTTTTCAGCAATGGAAAGAGA[C]GGAAGGAGATCC | SEQ ID No: 12 | ...[T]... |
| | | | GGCGTTTCTCCCT | | |
| 13 | rs1080985 | CYP2D6 | CCAGCCTGGACAACTTGGAAGAACC[G]GGTCTCTACAAA | SEQ ID No: 13 | ...[C]... |
| | | | AAATACAAAATTA | | |
| 14 | rs35742686 | CYP2D6 | GCTGGATGAGCTGCTAACTGAGCAC[-]GGATGACCTGGG | SEQ ID No: 14 | ...[A]... |
| | | | ACCCAGCCCAGCC | | |
| 15 | rs3892097 | CYP2D6 | CCCTTACCCGCATCTCCCACCCCCA[A]GACGCCCCTTTC | SEQ ID No: 15 | ...[G]... |
| | | | GCCCCAACGGTCT | | |
| 16 | rs5030655 | CYP2D6 | CCTGGGCAAGAAGTCGCTGGAGCAG[-]GGGTGACCGAGG | SEQ ID No: 16 | ...[T]... |
| | | | AGGCCGCCTGCCT | | |
| 17 | rs5030867 | CYP2D6 | TGGGGCCTCCTGCTCATGATCCTAC[C]YCCGGATGTGCA | SEQ ID No: 17 | ...[A]... |
| | | | GCSTGAGCCCATC | | |
| 18 | rs5030865 | CYP2D6 | TTTGTGCCCTTCTGCCCATCACCCAC[T]GGAGTGGTTGG | SEQ ID No: 18 | ...[A/C]... |
| | | | CGAAGGCGGCACAA | | |
| 19 | rs28371720 | CYP2D6 | TGAGGCCTTCCTGGCAGAGATGGAG[-]AGGTGAGAGTGG | SEQ ID No: 19 | ...[A/AGA]... |
| | | | CTGCCACGGTGGG | | |
| 20 | rs1065852 | CYP2D6 | GCGCCAACGCTGGGCTGCACGCTAC[T]CACCAGGCCCCC | SEQ ID No: 20 | ...[C]... |
| | | | TGCCACTGCCCGG | | |
| 21 | rs5030863 | CYP2D6 | AAGAGGCCCTGACCCTCCCTCTGCA[C]TTGCGGCGCCGC | SEQ ID No: 21 | ...[G]... |
| | | | TTCGGGGACGTGT | | |
| 22 | rs72549357 | CYP2D6 | TGTGTTCTGGAAGTCCACATGCAGC[A]GGTTGCCCAGCC | SEQ ID No: 22 | ...[-]... |
| | | | CGGGCAGTGGCAG | | |
| 23 | rs72549357 | CYP2D6 | TGTGTTCTGGAAGTCCACATGCAGC[AA]GGTTGCCCAGC | SEQ ID No: 23 | ...[-]... |
| | | | CCGGGCAGTGGCAG | | |
| 24 | rs28371706 | CYP2D6 | GCCGACCGCCCGCCTGTGCCCATCA[T]CCAGATCCTGGG | SEQ ID No: 24 | ...[C]... |
| | | | YTTYGGGCCGCGT | | |
| 25 | n/a*** | CYP2D6 | CCGGGGCTGTCCAGTGGGCAC[AGTGGGCAC]CGAGAAGC | SEQ ID No: 25 | ...[-]... |
| | | | TGAAGTGCTGCAG | | |
| 26 | rs59421388 | CYP2D6 | TCTGGTCGCCGCACCTGCCCTATCA[T]GTCGTCGATCKC | SEQ ID No: 26 | ...[C]... |
| | | | CTGTTGGACACGG | | |
| 27 | rs769258 | CYP2D6 | GCTAGAAGCACTGGTGCCCCTGGCC[A]TGATAGTGGCCA | SEQ ID No: 27 | ...[G]... |
| | | | TCTTCCTGCTCCT | | |
| 28 | rs16947 | CYP2D6 | GAGAACAGGTCAGCCACCACTATGC[A]CAGGTTCTCATC | SEQ ID No: 28 | ...[G]... |
| | | | ATTGAAGCTGCTC | | |
| 29 | rs1135840 | CYP2D6 | CATGGTGTCTTTGCTTTCCTGGTGA[C]CCCATCCCCCTA | SEQ ID No: 29 | ...[G]... |
| | | | TGAGCTTTGTGCT | | |
| 30 | rs28371725 | CYP2D6 | GGAAACAGTGCAGGGGCCGAGGGAG[A]AAGGGTACAGGC | SEQ ID No: 30 | ...[G]... |
| | | | GGGGGCCCATGAA | | |
| 31 | rs2740574 | CYP3A4 | GAGGACAGCCATAGAGACAAGGGCA[G]GAGAGAGGCGAT | SEQ ID No: 31 | ...[A]... |
| | | | TTAATAGATTTTA | | |
| 32 | rs776746 | CYP3A5 | CTTTAAAGAGCTCTTTTGTCTTTCA[A]TATCTCTTCCCT | SEQ ID No: 32 | ...[G]... |
| | | | GTTTGGACCACAT | | |
| 33 | rs9923231 | VKORC1 | GATTATAGGCGTGAGCCACCGCACC[A]GGCCAATGGTTG | SEQ ID No: 33 | ...[C/G/T]... |
| | | | TTTTTCAGGTCTT | | |
| 34 | rs104894539 | VKORC1 | AGTGCTCTCGCTCTACGCGCTGCAC[T]TGAAGGCGGCGC | SEQ ID No: 34 | ...[G]... |
| | | | GCGCCCGGGACCG | | |
| 35 | rs104894541 | VKORC1 | CAGCTGTTCGCGCGTCTTCTCCTCC[G]GGTGTGCACGGG | SEQ ID No: 35 | ...[A/C/T]... |
| | | | AGTGGGAGGCGTG | | |
| 36 | rs104894540 | VKORC1 | CGGGATTACCGCGCGCTCTGCGACG[C]GGGCACCGCCAT | SEQ ID No: 36 | ...[AG/T]... |
| | | | CAGCTGTTCGCGC | | |
| 37 | rs72547529 | VKORC1 | CTGTCCTGTCCCAGCACATGCTCCA[T]CAGCCCGAAACC | SEQ ID No: 37 | ...[C]... |
| | | | CCTGCCCCACCTG | | |
| 38 | n/a*** | VKORC1 | ATGGCGGTGCCCACGTCGCAGAGCG[A]GCGGTAATCCCG | SEQ ID No: 38 | ...[C]... |
| | | | GTCCCGGGCGCGC | | |
| 39 | rs762551 | CYP1A2 | TGCTCAAAGGGTGAGCTCTGTGGGC[A]CAGGACGCATGG | SEQ ID No.: 39 | ...[C]... |
| | | | TAGATGGAGCTTA | | |
| 40 | rs2257401 | CYP3A7 | TTCAGGGAGGAACTTCTCAGGCTCT[C]TCCAGTACTTTG | SEQ ID NO.: 40 | ...[G]... |
| | | | GGTCATGATGAAG | | |

*Unless otherwise indicated, the DNA context sequences are shown in FASTA format, as presented by NCBI within the rs cluster report for the SNP listed in the NCBI SNP reference database accessible at http://www.ncbi.nlm.nih.gov/snp.
**Brackets (i.e., “[...]”) appear within each context sequence to indicate the location (i.e., the “polymorphism marker” or “marker”) of the polymorphic region in the sequence.
***No corresponding rs cluster report is known to be listed in the NCBI SNP reference database for this polymorphism.

In Table 1, the active alleles are associated with “SNP markers” called “rs numbers” in the refSNP database. A SNP marker in dbSNP references a SNP cluster report identification number (i.e., a “rs number”) in the refSNP database. The context sequences shown in Table 1 include allelic variants identified as active for providing prognostic information according to the principles of the invention. The context sequences include the active polymorphism located in the polymorphic regions of the relevant genes. The context sequences also include a number of nucleotide bases flanking the active polymorphism SNP in the polymorphic region of the respective gene. In the context sequences shown in Table 1, the polymorphic region is shown within brackets (i.e., the “polymorphism marker” or “marker” of the polymorphic region in the sequence) in the context sequence for identification purposes.

Table 1 also show the rs cluster report number (i.e., the “rs number”) associated with the active polymorphism SNP in dbSNP maintained by NCBI, as well as other known SNP(s) for each rs number listed in Table 1. The other alleles not identified as “active alleles”, according to the principles of the invention, are also associated with the cluster reports for rs numbers in the refSNP database. Both the active and the inactive alleles from the cluster report for an rs number are shown in Table 1.

It has been determined that the active alleles identified in Table 1 are predictive of a differential metabolism of a patient as compared with an average patient metabolism not having an active allele. Certain active alleles are associated with being metabolically upregulating, in general, and others are associated with being metabolically down regulating, in general. In addition, the general metabolic characterizations of the active alleles are especially useful with determining prognostic information about a patient's metabolism with respect to various types of drugs. The prognostic information may be utilized to determine an appropriate therapy for the patient.

In Table 2 below, the general metabolic characterizations of the active alleles in Table 1 are listed. Table 2 also lists exemplary types of drugs in which a metabolic characterization of a patient may be utilized to determine prognostic information about treatment options for a patient. The exemplary drugs listed in Table 2 are shown as demonstrative only and are not meant to be limiting in determining potential treatment options for a patient according to the principles of the invention.

TABLE 2

| | | | Metabolic | | |
|---|---|---|---|---|---|
| No. | NCBI rs# | Gene | Characterization | SEQ ID | Exemplary Drug(s) |
| 1 | rs1799853 | CYP2C9 | downregulating | SEQ ID No: 1 | NSAIDs (e.g., celecoxib, ibuprofen, diclofenac), |
| | | | | | phenytoin, warfarin, etc. |
| 2 | rs1057910 | CYP2C9 | downregulating | SEQ ID No: 2 | NSAIDs (e.g., celecoxib, ibuprofen, diclofenac), |
| | | | | | phenytoin, warfarin, etc. |
| 3 | rs56165452 | CYP2C9 | downregulating | SEQ ID No: 3 | NSAIDs (e.g., celecoxib, ibuprofen, diclofenac), |
| | | | | | phenytoin, warfarin, etc. |
| 4 | rs28371686 | CYP2C9 | downregulating | SEQ ID No: 4 | NSAIDs (e.g., celecoxib, ibuprofen, diclofenac), |
| | | | | | phenytoin, warfarin, etc. |
| 5 | rs9332131 | CYP2C9 | downregulating | SEQ ID No: 5 | NSAIDs (e.g., celecoxib, ibuprofen, diclofenac), |
| | | | | | phenytoin, warfarin, etc. |
| 6 | rs4244285 | CYP2C19 | downregulating | SEQ ID No: 6 | Diazepam, cytlopram, sertraline, etc. |
| 7 | rs4986893 | CYP2C19 | downregulating | SEQ ID No: 7 | Diazepam, cytlopram, sertraline, etc. |
| 8 | rs28399504 | CYP2C19 | downregulating | SEQ ID No: 8 | Diazepam, cytlopram, sertraline, etc. |
| 9 | rs56337013 | CYP2C19 | downregulating | SEQ ID No: 9 | Diazepam, cytlopram, sertraline, etc. |
| 10 | rs72552267 | CYP2C19 | downregulating | SEQ ID No: 10 | Diazepam, cytlopram, sertraline, etc. |
| 11 | rs72558186 | CYP2C19 | downregulating | SEQ ID No: 11 | Diazepam, cytlopram, sertraline, etc. |
| 12 | rs41291556 | CYP2C19 | downregulating | SEQ ID No: 12 | Diazepam, cytlopram, sertraline, etc. |
| 13 | rs1080985 | CYP2D6 | downregulating | SEQ ID No: 13 | Opioids, tapentadol, amphetamines, tramadol, duloxetine, fluoxetine, |
| | | | | | paroxetine, venlafaxine, cyclobenzaprine, bupropion, etc. |
| 14 | rs35742686 | CYP2D6 | downregulating | SEQ ID No: 14 | Opioids, tapentadol, amphetamines, tramadol, duloxetine, fluoxetine, |
| | | | | | paroxetine, venlafaxine, cyclobenzaprine, bupropion, etc. |
| 15 | rs3892097 | CYP2D6 | downregulating | SEQ ID No: 15 | Opioids, tapentadol, amphetamines, tramadol, duloxetine, fluoxetine, |
| | | | | | paroxetine, venlafaxine, cyclobenzaprine, bupropion, etc. |
| 16 | rs5030655 | CYP2D6 | downregulating | SEQ ID No: 16 | Opioids, tapentadol, amphetamines, tramadol, duloxetine, fluoxetine, |
| | | | | | paroxetine, venlafaxine, cyclobenzaprine, bupropion, etc. |
| 17 | rs5030867 | CYP2D6 | downregulating | SEQ ID No: 17 | Opioids, tapentadol, amphetamines, tramadol, duloxetine, fluoxetine, |
| | | | | | paroxetine, venlafaxine, cyclobenzaprine, bupropion, etc. |
| 18 | rs5030865 | CYP2D6 | downregulating | SEQ ID No: 18 | Opioids, tapentadol, amphetamines, tramadol, duloxetine, fluoxetine, |
| | | | | | paroxetine, venlafaxine, cyclobenzaprine, bupropion, etc. |
| 19 | rs28371720 | CYP2D6 | downregulating | SEQ ID No: 19 | Opioids, tapentadol, amphetamines, tramadol, duloxetine, fluoxetine, |
| | | | | | paroxetine, venlafaxine, cyclobenzaprine, bupropion, etc. |
| 20 | rs1065852 | CYP2D6 | downregulating | SEQ ID No: 20 | Opioids, tapentadol, amphetamines, tramadol, duloxetine, fluoxetine, |
| | | | | | paroxetine, venlafaxine, cyclobenzaprine, bupropion, etc. |
| 21 | rs5030863 | CYP2D6 | downregulating | SEQ ID No: 21 | Opioids, tapentadol, amphetamines, tramadol, duloxetine, fluoxetine, |
| | | | | | paroxetine, venlafaxine, cyclobenzaprine, bupropion, etc. |
| 22 | rs72549357 | CYP2D6 | downregulating | SEQ ID No: 22 | Opioids, tapentadol, amphetamines, tramadol, duloxetine, fluoxetine, |
| | | | | | paroxetine, venlafaxine, cyclobenzaprine, bupropion, etc. |
| 23 | rs72549357 | CYP2D6 | downregulating | SEQ ID No: 23 | Opioids, tapentadol, amphetamines, tramadol, duloxetine, fluoxetine, |
| | | | | | paroxetine, venlafaxine, cyclobenzaprine, bupropion, etc. |
| 24 | rs28371706 | CYP2D6 | downregulating | SEQ ID No: 24 | Opioids, tapentadol, amphetamines, tramadol, duloxetine, fluoxetine, |
| | | | | | paroxetine, venlafaxine, cyclobenzaprine, bupropion, etc. |
| 25 | n/a*** | CYP2D6 | downregulating | SEQ ID No: 25 | Opioids, tapentadol, amphetamines, tramadol, duloxetine, fluoxetine, |
| | | | | | paroxetine, venlafaxine, cyclobenzaprine, bupropion, etc. |
| 26 | rs59421388 | CYP2D6 | downregulating | SEQ ID No: 26 | Opioids, tapentadol, amphetamines, tramadol, duloxetine, fluoxetine, |
| | | | | | paroxetine, venlafaxine, cyclobenzaprine, bupropion, etc. |
| 27 | rs769258 | CYP2D6 | normal | SEQ ID No: 27 | Opioids, tapentadol, amphetamines, tramadol, duloxetine, fluoxetine, |
| | | | | | paroxetine, venlafaxine, cyclobenzaprine, bupropion, etc. |
| 28 | rs16947 | CYP2D6 | upregulating | SEQ ID No: 28 | Opioids, tapentadol, amphetamines, tramadol, duloxetine, fluoxetine, |
| | | | | | paroxetine, venlafaxine, cyclobenzaprine, bupropion, etc. |
| 29 | rs1135840 | CYP2D6 | downregulating | SEQ ID No: 29 | Opioids, tapentadol, amphetamines, tramadol, duloxetine, fluoxetine, |
| | | | | | paroxetine, venlafaxine, cyclobenzaprine, bupropion, etc. |
| 30 | rs28371725 | CYP2D6 | downregulating | SEQ ID No: 30 | Opioids, tapentadol, amphetamines, tramadol, duloxetine, fluoxetine, |
| | | | | | paroxetine, venlafaxine, cyclobenzaprine, bupropion, etc. |
| 31 | rs2740574 | CYP3A4 | downregulating | SEQ ID No: 31 | Buprenorphine, methadone, buprenorphine, naloxone, |
| | | | | | meperidine, fentanyl, etc. |
| 32 | rs776746 | CYP3A5 | downregulating | SEQ ID No: 32 | Buprenorphine, methadone, buprenorphine, naloxone, |
| | | | | | meperidine, fentanyl, etc. |
| 33 | rs9923231 | VKORC1 | downregulating | SEQ ID No: 33 | NSAIDs (e.g., celecoxib, ibuprofen, diclofenac), |
| | | | | | phenytoin, warfarin, etc. |
| 34 | rs104894539 | VKORC1 | downregulating | SEQ ID No: 34 | NSAIDs (e.g., celecoxib, ibuprofen, diclofenac), |
| | | | | | phenytoin, warfarin, etc. |
| 35 | rs104894541 | VKORC1 | downregulating | SEQ ID No: 35 | NSAIDs (e.g., celecoxib, ibuprofen, diclofenac), |
| | | | | | phenytoin, warfarin, etc. |
| 36 | rs104894540 | VKORC1 | downregulating | SEQ ID No: 36 | NSAIDs (e.g., celecoxib, ibuprofen, diclofenac), |
| | | | | | phenytoin, warfarin, etc. |
| 37 | rs72547529 | VKORC1 | downregulating | SEQ ID No: 37 | NSAIDs (e.g., celecoxib, ibuprofen, diclofenac), |
| | | | | | phenytoin, warfarin, etc. |
| 38 | n/a*** | VKORC1 | downregulating | SEQ ID No: 38 | NSAIDs (e.g., celecoxib, ibuprofen, diclofenac), |
| | | | | | phenytoin, warfarin, etc. |
| 39 | rs762551 | CYP1A2 | upregulating | SEQ ID No.: 39 | Any of the exemplary drugs listed above |
| 40 | rs2257401 | CYP3A7 | upregulating | SEQ ID No.: 40 | Any of the exemplary drugs listed above |

***No corresponding rs cluster report is known to be listed in the NCBI SNP reference database for this polymorphism.

The invention further provides systems and methods which are associated, at least in part, with a determination of the presence or absence of one or more of the active alleles listed above in Tables 1 & 2. For example, information obtained using the diagnostic assays described herein is useful for determining how a subject will likely metabolize a drug if administered the medication and/or experience an positive response to the treatment. Based on this prognostic information, a clinician can recommend a therapeutic protocol useful for treating an individual, or adjust a previously administered therapy to accommodate the patient's sensitivities.

In addition, knowledge of the identity of a particular allelic variant in an individual's genetic profile allows customization of medication therapy based on the particular individual's genetic profile. For example, an individual's genetic profile can enable a doctor: 1) to more effectively prescribe a drug that will address the patient's medical condition; 2) to better determine an appropriate dosage of a particular drug and 3) to identify novel targets for drug development. The ability to target populations expected to show the highest clinical benefit, based on the genetic profile, can enable: 1) the repositioning of marketed drugs with disappointing market results; 2) the rescue of drug candidates whose clinical development has been discontinued as a result of safety or efficacy limitations, which may be patient subgroup-specific; and 3) an accelerated and less costly development for drug candidates and more optimal drug labeling.

Detection of point mutations or other types of allelic variants can be accomplished by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic region and having, for example, about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

In other detection methods, it is necessary to first amplify at least a portion of the gene of interest prior to identifying the allelic variant. Amplification can be performed, e.g., by PCR, according to methods known in the art. In one embodiment, genomic DNA of a cell is exposed to two PCR primers and amplification for a number of cycles sufficient to produce the required amount of amplified DNA. Alternative amplification methods include: self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In other embodiments, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci. USA 74:560 or Sanger et al. (1977) Proc. Nat. Acad. Sci. 74:5463. It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, analyses where only one nucleotide is detected can be carried out.

Yet other sequencing methods are disclosed, e.g., in U.S. Pat. No. 5,580,732 entitled “Method of DNA Sequencing Employing a Mixed DNA-Polymer Chain Probe” and U.S. Pat. No. 5,571,676 entitled “Method For Mismatch-Directed In Vitro DNA Sequencing.” In some cases, the presence of a specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility are used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; Cotton (1993) Mutat. Res. 285:125-144 and Hayashi (1992) Genet. Anal. Tech. Appl. 9:73-79). Single-stranded DNA fragments of sample and control nucleic acids are denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In other embodiments, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Exemplary techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of an allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on the OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase or horseradish peroxidase. This permits the detection of the two alleles using a high throughput format that leads to the production of two different colors.

The invention further provides methods for detecting a polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such polymorphisms.

In one embodiment, the polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide is present in the polymorphic site of the target molecule was complementary to that of the nucleotide derivative used in the reaction. This method has the advantage that it does not require the determination of large amounts of extraneous sequence data.

In another embodiment, a solution-based method is used for determining the identity of the nucleotide of the polymorphic site. Cohen et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al., supra, is preferably a heterogeneous phase assay, in which the primer or the target molecule is immobilized to a solid phase.

Several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

The genotype information obtained from analyzing a sample of a patient's genetic material may be utilized, according to the principles of the invention, to predict whether a patient has a level of risk associated with taking a medication for treating medical condition. The risk may be associated with a side effect the patient may be susceptible to developing, an efficacy of the drug to the patient specifically or some combination thereof.

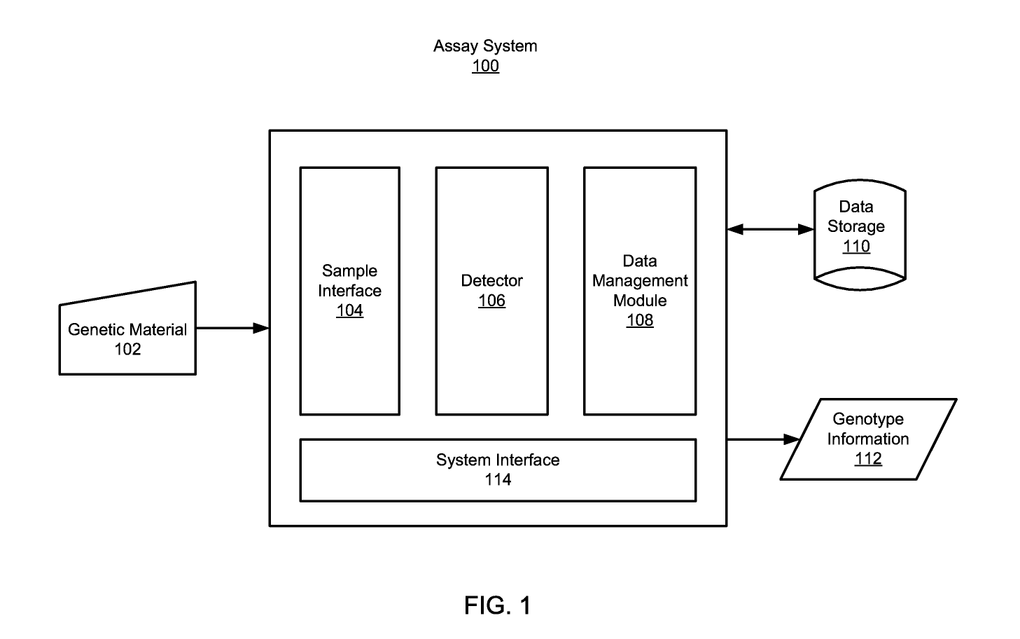

Referring to FIG. 1, depicted is an assay system 100. An assay system, such as assay system 100, may access or receive a genetic material, such as genetic material 102. The sample of genetic material 102 can be obtained from a patient by any suitable manner. The sample can be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Table I above. The genotype information 112 may be stored in data storage 110 or transmitted to another system or entity via a system interface 114.

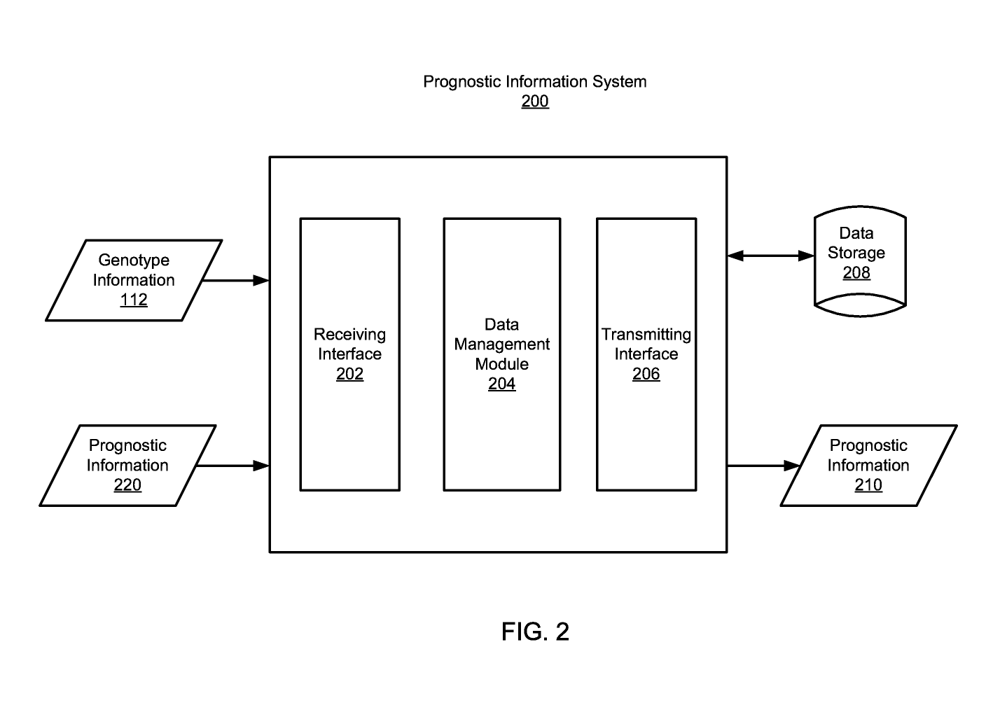

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

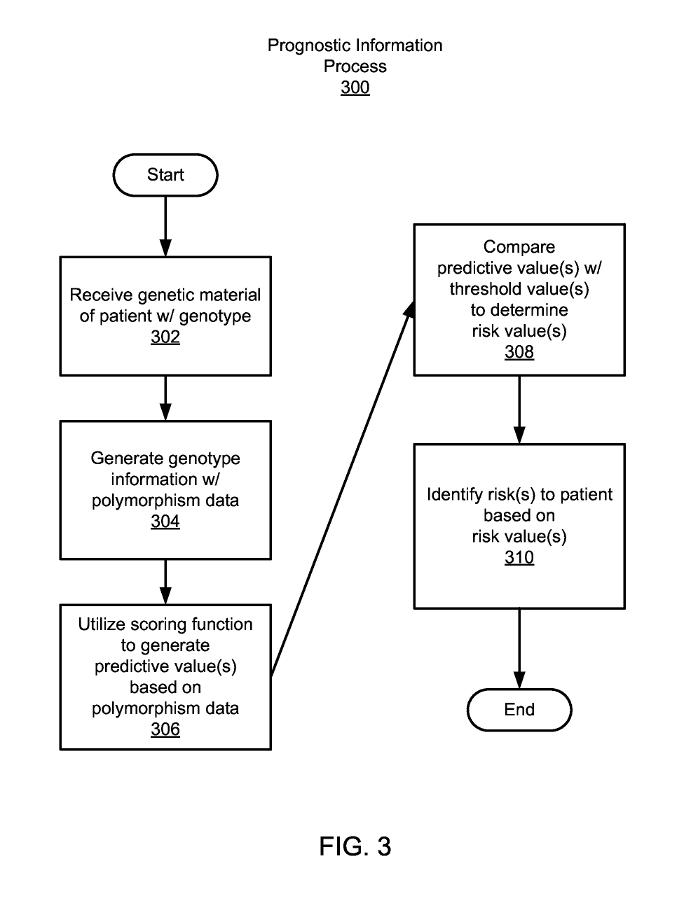

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Table 1, and metabolism activity associated with the DNA polymorphisms described above in Table 2. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign the predictive values.

For example, an additive effect algorithm may be utilized to generate an analysis of a patient's genetic predisposition to properly or improperly metabolize a drug which might be prescribed for treating a medical condition associated with the patient. In the additive effect algorithm, polymorphism data in the genotype information obtained from analyzing a patient's genetic material is utilized to indicate how active alleles are identified from a patient's genotype information. A tested active allele may be (1) absent or present in either (2) a heterozygous or (3) a homozygous variant in the patient's genotype. According to the additive effect algorithm, the allelic variants identified from a patient's genotype information is assigned an integer value depending on how it appears in the patient's genotype, for example, by assigning a value of “3” if an allelic variant appears as homozygous for an active allele, a value of “2” if it appears as heterozygous for the active allele and a value of “1” if the active allelic variant is determined to be absent from the patient's genotype. The values generated are a form of prognostic information 210.

At step 308, a sum of the assigned predictive values (e.g., 1, 2 or 3) from all the alleles tested forms an aggregate value which is called an additive effect risk index score. This score can be used to help assess risk factors associated with prescribing a medication to a patient. The risk index score may be evaluated by comparing the generated score with a predetermined threshold value to determine a risk value.

At step 310, the result of the comparison obtained in step 308 generates a second form of prognostic information 220. For example, (a) if the determined sum is higher than the threshold value, it can be predicted that the patient is at a higher risk for a negative risk factor associated with prescribing the patient the medication; (b) if the determined sum is at or near the threshold value, it can be predicted that the patient is at a moderate risk for negative risk factors associated with prescribing the patient the medication; and (c) if the determined sum is below the threshold value, it can be predicted that the patient is at a low risk for negative risk factors associated with prescribing the patient the medication.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300.

Other algorithms may also be used in a similar manner to generate useful forms of prognostic information for determining treatment options for a patient. For example, a more complex Genetic Predisposition Analysis algorithm may be utilized which assigns predetermined values based upon the presence or absence of select active alleles in a patient's genotype information. The predetermined values may be the same for all drugs in a potential therapeutic regimen. Or predetermined values may be different, such as by being dependent on the therapeutic compounds being considered as a treatment for a patient. For example, the predetermined values may be associated a specific polymorphisms and one or more specific medications. Table 2 shown above, provides specific associations between specific polymorphisms and certain drugs or types of drugs, according to an exemplary embodiment. According to an embodiment, a specific polymorphism can be assigned a numerical value, such as 1, 2, 3 or 4, for a medication. All the assigned values can be summed and/or averaged to form an aggregate value associates with a patient's genotype information and one or more proposed medications. The predetermined values may be combined in various ways to form summary information about a patient, such as a Medication Metabolism Metric (M3). The summary information may be used to provide a simplified decision making process in considering prognostic information about treatment options for a patient generated according to the principles of the invention.

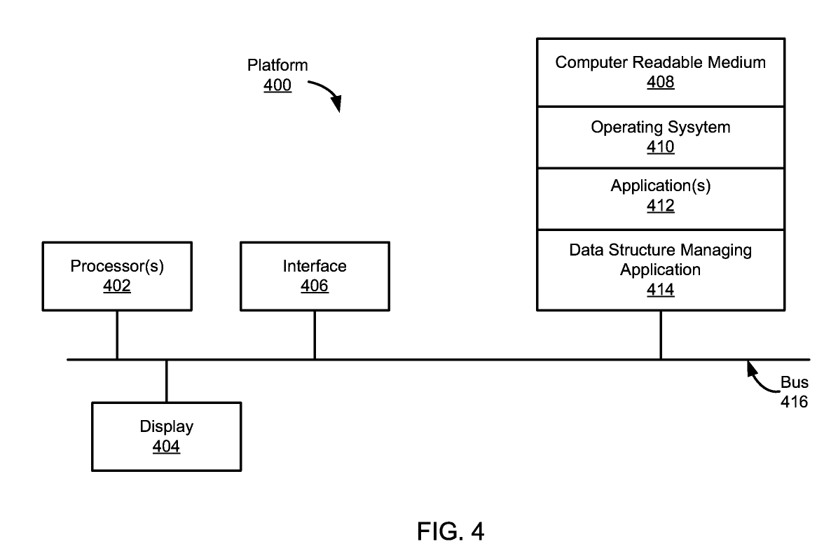

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

What is claimed is:

1. A system for performing an assay, comprising:
a sample interface configured to present a sample of human genetic material of a patient associated with having a medical condition, the patient having a genotype; and
a detector configured for detecting in the sample a presence of at least five polymorphisms in the genotype to determine an assay result comprising data describing a presence or an absence of the polymorphisms, wherein the polymorphisms are selected from a group consisting of:
a T allele at the marker of SEQ ID No: 1 in the CYP2C9 gene,
a C allele at the marker of SEQ ID No: 2 in the CYP2C9 gene,
a C allele at the marker of SEQ ID No: 3 in the CYP2C9 gene,
a G allele at the marker of SEQ ID No: 4 in the CYP2C9 gene,
a deletion allele at the marker of SEQ ID No: 5 in the CYP2C9 gene,
an A allele at the marker of SEQ ID No: 6 in the CYP2C19 gene,
an A allele at the marker of SEQ ID No: 7 in the CYP2C19 gene,
a G allele at the marker of SEQ ID No: 8 in the CYP2C19 gene,
a T allele at the marker of SEQ ID No: 9 in the CYP2C19 gene,
an A allele at the marker of SEQ ID No: 10 in the CYP2C19 gene,
an A allele at the marker of SEQ ID No: 11 in the CYP2C19 gene,
a C allele at the marker of SEQ ID No: 12 in the CYP2C19 gene,
a G allele at the marker of SEQ ID No: 13 in the CYP2D6 gene,
a deletion allele at the marker of SEQ ID No: 14 in the CYP2D6 gene,
an A allele at the marker of SEQ ID No: 15 in the CYP2D6 gene,
a deletion allele at the marker of SEQ ID No: 16 in the CYP2D6 gene,
a C allele at the marker of SEQ ID No: 17 in the CYP2D6 gene,
a T allele at the marker of SEQ ID No: 18 in the CYP2D6 gene,
a deletion allele at the marker of SEQ ID No: 19 in the CYP2D6 gene,
a T allele at the marker of SEQ ID No: 20 in the CYP2D6 gene,
a C allele at the marker of SEQ ID No: 21 in the CYP2D6 gene,
an A allele at the marker of SEQ ID No: 22 in the CYP2D6 gene,
an AA allele at the marker of SEQ ID No: 23 in the CYP2D6 gene,
a T allele at the marker of SEQ ID No: 24 in the CYP2D6 gene,
a AGTGGGCAC allele at the marker of SEQ ID No: 25 in the CYP2D6 gene,
a T allele at the marker of SEQ ID No: 26 in the CYP2D6 gene,
an A allele at the marker of SEQ ID No: 27 in the CYP2D6 gene,
an A allele at the marker of SEQ ID No: 28 in the CYP2D6 gene,
a C allele at the marker of SEQ ID No: 29 in the CYP2D6 gene,
an A allele at the marker of SEQ ID No: 30 in the CYP2D6 gene,
a G allele at the marker of SEQ ID No: 31 in the CYP3A4 gene,
an A allele at the marker of SEQ ID No: 32 in the CYP3A5 gene,
an A allele at the marker of SEQ ID No: 33 in the VKORC1 gene,
a T allele at the marker of SEQ ID No: 34 in the VKORC1 gene,
a G allele at the marker of SEQ ID No: 35 in the VKORC1 gene,
a C allele at the marker of SEQ ID No: 36 in the VKORC1 gene,
a T allele at the marker of SEQ ID No: 37 in the VKORC1 gene,
an A allele at the marker of SEQ ID No: 38 in the VKORC1 gene,
an A allele at the marker of SEQ ID No: 39 in the CYP1A2 gene, and
a C allele at the marker of SEQ ID No: 40 in the CYP3A7 gene.

2. The system of claim 1, wherein the detector is configured to test for detecting a presence of at least ten polymorphisms selected from the group.

3. The system of claim 1, wherein the detector is configured to test for detecting a presence of at least twenty polymorphisms selected from the group.

4. The system of claim 1, wherein the detector is configured to test for detecting a presence of at least thirty polymorphisms selected from the group.

5. The system of claim 1, further comprising
a data management module configured to generate, utilizing a processor, genotype information associated with the polymorphisms.

6. The system of claim 1, wherein the detector is configured to utilize at least one of: allele specific hybridization, allele specific oligonucleotide ligation, primer extension, mini-sequencing, mass spectroscopy, hetero-duplex analysis, single strand conformational polymorphism, denaturing gradient gel electrophoresis, oligonucleotide microarray analysis, temperature gradient gel electrophoresis and combinations thereof.

7. The system of claim 1, wherein the detector is configured to detect for the presence of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, a DNA fragment thereof, a homologous DNA sequence thereof having at least 50% homology, and combinations thereof.

8. A system for preparing prognostic information, comprising:
a receiving interface configured to receive genotype information comprising data indicating a presence or an absence of at least five polymorphisms in a genotype of a patient associated with having a medical condition, wherein the polymorphisms are selected from a group consisting of:
a T allele at the marker of SEQ ID No: 1 in the CYP2C9 gene,
a C allele at the marker of SEQ ID No: 2 in the CYP2C9 gene,
a C allele at the marker of SEQ ID No: 3 in the CYP2C9 gene,
a G allele at the marker of SEQ ID No: 4 in the CYP2C9 gene,
a deletion allele at the marker of SEQ ID No: 5 in the CYP2C9 gene,
an A allele at the marker of SEQ ID No: 6 in the CYP2C19 gene,
an A allele at the marker of SEQ ID No: 7 in the CYP2C19 gene,
a G allele at the marker of SEQ ID No: 8 in the CYP2C19 gene,
a T allele at the marker of SEQ ID No: 9 in the CYP2C19 gene,
an A allele at the marker of SEQ ID No: 10 in the CYP2C19 gene,
an A allele at the marker of SEQ ID No: 11 in the CYP2C19 gene,
a C allele at the marker of SEQ ID No: 12 in the CYP2C19 gene,
a G allele at the marker of SEQ ID No: 13 in the CYP2D6 gene,
a deletion allele at the marker of SEQ ID No: 14 in the CYP2D6 gene,
an A allele at the marker of SEQ ID No: 15 in the CYP2D6 gene,
a deletion allele at the marker of SEQ ID No: 16 in the CYP2D6 gene,
a C allele at the marker of SEQ ID No: 17 in the CYP2D6 gene,
a T allele at the marker of SEQ ID No: 18 in the CYP2D6 gene,
a deletion allele at the marker of SEQ ID No: 19 in the CYP2D6 gene,
a T allele at the marker of SEQ ID No: 20 in the CYP2D6 gene,
a C allele at the marker of SEQ ID No: 21 in the CYP2D6 gene,
an A allele at the marker of SEQ ID No: 22 in the CYP2D6 gene,
an AA allele at the marker of SEQ ID No: 23 in the CYP2D6 gene,
a T allele at the marker of SEQ ID No: 24 in the CYP2D6 gene,
a AGTGGGCAC allele at the marker of SEQ ID No: 25 in the CYP2D6 gene,
a T allele at the marker of SEQ ID No: 26 in the CYP2D6 gene,
an A allele at the marker of SEQ ID No: 27 in the CYP2D6 gene,
an A allele at the marker of SEQ ID No: 28 in the CYP2D6 gene,
a C allele at the marker of SEQ ID No: 29 in the CYP2D6 gene,
an A allele at the marker of SEQ ID No: 30 in the CYP2D6 gene,
a G allele at the marker of SEQ ID No: 31 in the CYP3A4 gene,
an A allele at the marker of SEQ ID No: 32 in the CYP3A5 gene,
an A allele at the marker of SEQ ID No: 33 in the VKORC1 gene,
a T allele at the marker of SEQ ID No: 34 in the VKORC1 gene,
a G allele at the marker of SEQ ID No: 35 in the VKORC1 gene,
a C allele at the marker of SEQ ID No: 36 in the VKORC1 gene,
a T allele at the marker of SEQ ID No: 37 in the VKORC1 gene,
an A allele at the marker of SEQ ID No: 38 in the VKORC1 gene,
an A allele at the marker of SEQ ID No: 39 in the CYP1A2 gene, and
a C allele at the marker of SEQ ID No: 40 in the CYP3A7 gene; and
a data management module configured to generate, utilizing a processor, the prognostic information comprising at least one predictive value(s) associated with the patient and their treatment with a medication associated with addressing the patient's medical condition, wherein the predictive value(s) correspond with respective polymorphisms selected from the group.

9. The system of claim 8, wherein the receiving interface is configured to receive genotype information comprising data indicating the presence or absence of at least six polymorphisms selected from the group.

10. The system of claim 8, wherein the receiving interface is configured to receive genotype information comprising data indicating the presence or absence of at least eight polymorphisms selected from the group.

11. The system of claim 8, wherein the receiving interface is configured to receive genotype information comprising data indicating the presence or absence of at least ten polymorphisms selected from the group.

12. The system claim 8, wherein the data management module is configured to generate the prognostic information utilizing a scoring function to determine the predictive value(s) based on the indicated presence or absence of the polymorphisms.

13. The system of claim 8, wherein the data management module is configured to determine the predictive value(s) based on the indicated presence or absence of the polymorphisms being homozygous or heterozygous.

14. The system of claim 8, wherein the data management module is configured to generate the prognostic information by adding the predictive value(s) to determine at least one aggregate value(s).

15. The system of claim 14, wherein the data management module is configured to generate the prognostic information by comparing the determined aggregate value(s) with at least one threshold value(s) to determine at least one risk value(s) associated with the patient.

16. A system for utilizing prognostic information, comprising:
a receiving interface configured to receive prognostic information associated with genotype information comprising data indicating a presence or an absence of at least five polymorphisms in a genotype of a patient associated with having a medical condition, wherein the polymorphisms are selected from a group consisting of:
a T allele at the marker of SEQ ID No: 1 in the CYP2C9 gene,
a C allele at the marker of SEQ ID No: 2 in the CYP2C9 gene,
a C allele at the marker of SEQ ID No: 3 in the CYP2C9 gene,
a G allele at the marker of SEQ ID No: 4 in the CYP2C9 gene,
a deletion allele at the marker of SEQ ID No: 5 in the CYP2C9 gene,
an A allele at the marker of SEQ ID No: 6 in the CYP2C19 gene,
an A allele at the marker of SEQ ID No: 7 in the CYP2C19 gene,
a G allele at the marker of SEQ ID No: 8 in the CYP2C19 gene,
a T allele at the marker of SEQ ID No: 9 in the CYP2C19 gene,
an A allele at the marker of SEQ ID No: 10 in the CYP2C19 gene,
an A allele at the marker of SEQ ID No: 11 in the CYP2C19 gene,
a C allele at the marker of SEQ ID No: 12 in the CYP2C19 gene,
a G allele at the marker of SEQ ID No: 13 in the CYP2D6 gene,
a deletion allele at the marker of SEQ ID No: 14 in the CYP2D6 gene,
an A allele at the marker of SEQ ID No: 15 in the CYP2D6 gene,
a deletion allele at the marker of SEQ ID No: 16 in the CYP2D6 gene,
a C allele at the marker of SEQ ID No: 17 in the CYP2D6 gene,
a T allele at the marker of SEQ ID No: 18 in the CYP2D6 gene,
a deletion allele at the marker of SEQ ID No: 19 in the CYP2D6 gene,
a T allele at the marker of SEQ ID No: 20 in the CYP2D6 gene,
a C allele at the marker of SEQ ID No: 21 in the CYP2D6 gene,
an A allele at the marker of SEQ ID No: 22 in the CYP2D6 gene,
an AA allele at the marker of SEQ ID No: 23 in the CYP2D6 gene,
a T allele at the marker of SEQ ID No: 24 in the CYP2D6 gene,
a AGTGGGCAC allele at the marker of SEQ ID No: 25 in the CYP2D6 gene,
a T allele at the marker of SEQ ID No: 26 in the CYP2D6 gene,
an A allele at the marker of SEQ ID No: 27 in the CYP2D6 gene,
an A allele at the marker of SEQ ID No: 28 in the CYP2D6 gene,
a C allele at the marker of SEQ ID No: 29 in the CYP2D6 gene,
an A allele at the marker of SEQ ID No: 30 in the CYP2D6 gene,
a G allele at the marker of SEQ ID No: 31 in the CYP3A4 gene,
an A allele at the marker of SEQ ID No: 32 in the CYP3A5 gene,
an A allele at the marker of SEQ ID No: 33 in the VKORC1 gene,
a T allele at the marker of SEQ ID No: 34 in the VKORC1 gene,
a G allele at the marker of SEQ ID No: 35 in the VKORC1 gene,
a C allele at the marker of SEQ ID No: 36 in the VKORC1 gene,
a T allele at the marker of SEQ ID No: 37 in the VKORC1 gene,
an A allele at the marker of SEQ ID No: 38 in the VKORC1 gene,
an A allele at the marker of SEQ ID No: 39 in the CYP1A2 gene, and
a C allele at the marker of SEQ ID No: 40 in the CYP3A7 gene; and
a data management module configured to utilize the received prognostic information to identify, utilizing a processor, at least one risk value(s) associated with the patient and their treatment with a medication associated addressing the patient's medical condition.

17. The system of claim 16, wherein the receiving interface is configured to receive prognostic information associated with genotype information comprising data indicating the presence or absence of at least six polymorphisms selected from the group.

18. The system of claim 16, wherein the receiving interface is configured to receive prognostic information associated with genotype information comprising data indicating the presence or absence of at least eight polymorphisms selected from the group.

19. The system of claim 16, wherein the receiving interface is configured to receive prognostic information associated with genotype information comprising data indicating the presence or absence of at least ten polymorphisms selected from the group.

20. The system of claim 16, wherein the data management module is configured to compare the received prognostic information with a medical record associated with the patient.


SEQUENCE LISTING (40 sequences)

SEQ ID NO: 1 - DNA, 51 bp, Homo sapiens
gatggggaagaggagcattgaggacygtgttcaagaggaagcccgctgcct

SEQ ID NO: 2 - DNA, 51 bp, Homo sapiens
tgtggtgcacgaggtccagagatacmttgaccttctccccaccagcctgcc

SEQ ID NO: 3 - DNA, 51 bp, Homo sapiens
gtggtgcacgaggtccagagatacahtgaccttctccccaccagcctgccc

SEQ ID NO: 4 - DNA, 51 bp, Homo sapiens
tgcacgaggtccagagatacattgascttctccccaccagcctgccccatg

SEQ ID NO: 5 - DNA, 51 bp, Homo sapiens
  misc_feature (25)..(25): May or may not be present
gattgcttcctgatgaaaatggagaaggtaaaatgtaaacaaaagcttagt

SEQ ID NO: 6 - DNA, 51 bp, Homo sapiens
ttcccactatcattgattatttcccvggaacccataacaaattacttaaaa

SEQ ID NO: 7 - DNA, 51 bp, Homo sapiens
acatcaggattgtaagcaccccctgratccaggtaaggccaagttttttgc

SEQ ID NO: 8 - DNA, 51 bp, Homo sapiens
gtcttaacaagaggagaaggcttcartggatccttttgtggtccttgtgct

SEQ ID NO: 9 - DNA, 51 bp, Homo sapiens
cctatgtttgttattttcaggaaaayggatttgtgtgggagagggcctggc

SEQ ID NO: 10 - DNA, 51 bp, Homo sapiens
cggcgtttctccctcatgackctgcrgaattttgggatggggaagaggagc

SEQ ID NO: 11 - DNA, 51 bp, Homo sapiens
tgcttcctgatcaaaatggagaaggwaaaatgttaacaaaagcttagttat

SEQ ID NO: 12 - DNA, 51 bp, Homo sapiens
aatcgttttcagcaatggaaagagayggaaggagatccggcgtttctccct

SEQ ID NO: 13 - DNA, 51 bp, Homo sapiens
ccagcctggacaacttggaagaaccsggtctctacaaaaaatacaaaatta

SEQ ID NO: 14 - DNA, 51 bp, Homo sapiens
  misc_feature (26)..(26): May or may not be present
gctggatgagctgctaactgagcacaggatgacctgggacccagcccagcc

SEQ ID NO: 15 - DNA, 51 bp, Homo sapiens
cccttacccgcatctcccacccccargacgcccctttcgccccaacggtct

SEQ ID NO: 16 - DNA, 51 bp, Homo sapiens
  misc_feature (26)..(26): May or may not be present
cctgggcaagaagtcgctggagcagtgggtgaccgaggaggccgcctgcct

SEQ ID NO: 17 - DNA, 51 bp, Homo sapiens
tggggcctcctgctcatgatcctacmyccggatgtgcagcstgagcccatc

SEQ ID NO: 18 - DNA, 52 bp, Homo sapiens
tttgtgcccttctgcccatcacccachggagtggttggcgaaggcggcacaa

SEQ ID NO: 19 - DNA, 53 bp, Homo sapiens
  misc_feature (26)..(28): May or may not be present
tgaggccttcctggcagagatggagagaaggtgagagtggctgccacggtggg

SEQ ID NO: 20 - DNA, 51 bp, Homo sapiens
gcgccaacgctgggctgcacgctacycaccaggccccctgccactgcccgg

SEQ ID NO: 21 - DNA, 51 bp, Homo sapiens
aagaggccctgaccctccctctgcasttgcggcgccgcttcggggacgtgt

SEQ ID NO: 22 - DNA, 51 bp, Homo sapiens
  misc_feature (26)..(26): May or may not be present
tgtgttctggaagtccacatgcagcaggttgcccagcccgggcagtggcag

SEQ ID NO: 23 - DNA, 52 bp, Homo sapiens
  misc_feature (26)..(27): This region may or may not be present in its entirety
tgtgttctggaagtccacatgcagcaaggttgcccagcccgggcagtggcag

SEQ ID NO: 24 - DNA, 51 bp, Homo sapiens
gccgaccgcccgcctgtgcccatcayccagatcctgggyttygggccgcgt

SEQ ID NO: 25 - DNA, 51 bp, Homo sapiens
  misc_feature (22)..(30): This region may or may not be present in its entirety
ccggggctgtccagtgggcacagtgggcaccgagaagctgaagtgctgcag

SEQ ID NO: 26 - DNA, 51 bp, Homo sapiens
tctggtcgccgcacctgccctatcaygtcgtcgatckcctgttggacacgg

SEQ ID NO: 27 - DNA, 51 bp, Homo sapiens
gctagaagcactggtgcccctggccrtgatagtggccatcttcctgctcct

SEQ ID NO: 28 - DNA, 51 bp, Homo sapiens
gagaacaggtcagccaccactatgcrcaggttctcatcattgaagctgctc

SEQ ID NO: 29 - DNA, 51 bp, Homo sapiens
catggtgtctttgctttcctggtgascccatccccctatgagctttgtgct

SEQ ID NO: 30 - DNA, 51 bp, Homo sapiens
ggaaacagtgcaggggccgagggagraagggtacaggcgggggcccatgaa

SEQ ID NO: 31 - DNA, 51 bp, Homo sapiens
gaggacagccatagagacaagggcargagagaggcgatttaatagatttta

SEQ ID NO: 32 - DNA, 51 bp, Homo sapiens
ctttaaagagctcttttgtctttcartatctcttccctgtttggaccacat

SEQ ID NO: 33 - DNA, 51 bp, Homo sapiens
  modified_base (26)..(26): a, c, t or g
gattataggcgtgagccaccgcaccnggccaatggttgtttttcaggtctt

SEQ ID NO: 34 - DNA, 51 bp, Homo sapiens
agtgctctcgctctacgcgctgcacktgaaggcggcgcgcgcccgggaccg

SEQ ID NO: 35 - DNA, 51 bp, Homo sapiens
  modified_base (26)..(26): a, c, t or g
cagctgttcgcgcgtcttctcctccnggtgtgcacgggagtgggaggcgtg

SEQ ID NO: 36 - DNA, 52 bp, Homo sapiens
  misc_feature (26)..(27): This region may encompass “c,” “ag,” or “t” wherein some positions may be absent
cgggattaccgcgcgctctgcgacgnngggcaccgccatcagctgttcgcgc

SEQ ID NO: 37 - DNA, 51 bp, Homo sapiens
ctgtcctgtcccagcacatgctccaycagcccgaaacccctgccccacctg

SEQ ID NO: 38 - DNA, 51 bp, Homo sapiens
atggcggtgcccacgtcgcagagcgmgcggtaatcccggtcccgggcgcgc

SEQ ID NO: 39 - DNA, 51 bp, Homo sapiens
tgctcaaagggtgagctctgtgggcmcaggacgcatggtagatggagctta

SEQ ID NO: 40 - DNA, 51 bp, Homo sapiens
ttcagggaggaacttctcaggctctstccagtactttgggtcatgatgaag